(19) United States
(12) Patent Application Publication
(10) Pub. No.: US 2023/0130473 A1
(43) Pub. Date: Apr. 27, 2023

(54) NEEDLE ASSEMBLY FOR FORMING HOLE THROUGH BIOLOGICAL WALL

(71) Applicant: Boston Scientific Medical Device Limited, Galway (IE)

(72) Inventors: Christian Balkovec, Burlington (CA); Gareth Davies, Toronto (CA); Yun Uhm, North York (CA)

(21) Appl. No.: 17/910,551

(22) PCT Filed: Mar. 15, 2021
(86) PCT No.: PCT/IB2021/052140

§ 371 Date: Sep. 9, 2022

Related U.S. Application Data
(60) Provisional application No. 62/992,215, filed on Mar. 20, 2020.

(51) Int. Cl. A61B 17/00; A61B 17/34

(52) U.S. Cl. CPC A61B 17/00234; A61B 17/34

(57) ABSTRACT

A needle assembly is configured to be movable into a cavity of a patient having a biological wall. A distal tip section extends from the needle assembly. The distal tip section is configured to form a pass-through hole extending through the biological wall of the patient (as, or while, the needle assembly is urged to move toward the biological wall). The distal tip section is also configured to prevent (at least in part) the removal of a free-floating tissue core from the biological wall as the pass-through hole is formed by the distal tip section.

### TECHNICAL FIELD

This document relates to the technical field of (and is not limited to) a needle assembly configured to be movable into a patient having a biological wall, and the needle assembly configured to form a pass-through hole extending through the biological wall of the patient (and method therefor).

### BACKGROUND

A medical needle assembly is a medical tool configured to pass through a biological wall of a patient, and may or may not include a passageway extending along a length of the medical needle assembly.

### SUMMARY

It will be appreciated that there exists a need to mitigate (at least in part) at least one problem associated with the existing needle assemblies (also called the existing technology). After much study of, and experimentation with, the existing needle assemblies, an understanding (at least in part) of the problem and its solution have been identified (at least in part) and are articulated (at least in part) as follows:

Radiofrequency needles are commonly used for puncturing the interatrial septum in transseptal catheterization procedures of the heart. They act by vaporizing a target tissue when radiofrequency energy is delivered through an active electrode at the distal tip. This is in opposition to mechanical needles which puncture the interatrial septum using mechanical force delivered by a user. While radiofrequency needles require less input force to puncture, have improved precision for puncture location, and reduce the risk of inadvertent mechanical puncture (due to their blunt tip compared to mechanical needles), they may remove (or reduce) some functionality for the user.

Mechanical needles used for puncturing the interatrial septum are typically characterized by having a hollow or open lumen. This open lumen allows for contrast delivery directly onto the site targeted for puncture to provide visual confirmation under fluoroscopy imaging for the user. Further, the open lumen allows for pressure measurements to be performed, enabling the user to confirm the area of the heart that they are in. Finally, the open lumen facilitates the anchoring of guidewires to be placed through the needle, and anchor (affix) the puncture location spanning from the right atrium to the left atrium in the heart of a patient.

A radiofrequency needle with an open lumen would deliver the above functionality to users accustomed to mechanical needles while still retaining the benefits of radiofrequency-based puncture of the interatrial septum. One issue, however, is the prospect of “coring” tissue when puncturing. An electrically active open lumen is characterized by a closed pathway of conductive material that vaporizes the tissue it encounters. Inside of the perimeter formed by the closed conductive pathway however, tissue is not vaporized but instead becomes separated from the larger wall of tissue it was a part of since all tissue surrounding it is vaporized. This action is similar to how a hole punch creates (forms) a separate disc of paper from a larger piece. Having a free-floating core of tissue in the bloodstream is highly undesirable as it presents a real risk to causing stroke or pulmonary embolism in a patient. Any open-lumen radiofrequency needle, therefore, would need a method to prevent this from occurring.

Known radiofrequency transseptal needles are commonly used as are mechanical transseptal needles. There are obvious advantages and disadvantages to both when examined from the lens of the puncture modality and the lumen being open or closed. Ideally, a product that marries the benefits of an open lumen with a radiofrequency-puncture modality may combine the advantages offered currently by both mechanical and radiofrequency transseptal needles. Open lumen RF delivery device for puncture of the fossa ovalis in the heart is well known by those skilled in the art.

FIG. 1 depicts a cross-sectional view of an embodiment of a known needle assembly having a known distal tip section.

Referring to the embodiment as depicted in FIG. 1, the known needle assembly defines a known lumen extending a length of the known needle assembly. The known needle assembly is configured to be movable into a cavity of a patient having a biological wall (an internal biological wall). The known distal tip section extends (distally) from the known needle assembly. The known distal tip section is configured to form a pass-through hole extending through the biological wall of the patient as (while) the known needle assembly is urged to move toward the biological wall. The known distal tip section is configured to form a free-floating tissue core (that is depicted in the known embodiment of FIG. 1) from a biological wall as (or while) a pass-through hole is formed by the distal tip section in response to moving the known needle assembly toward the biological wall. The known distal tip section is configured to form the free-floating tissue core that may result by assistance from, or the presence of, the entrance of the known lumen from the biological wall. As the known distal tip section is made to pass through the biological wall, the entrance of the known lumen cuts and forms the free-floating tissue core.

It may be a disadvantage (or dangerous) to form the free-floating tissue core within the patient. For instance, the free-floating tissue core may be formed in the heart of the patient, which then may be free to flow through the circulation system to the brain and cause a stroke, etc.

Therefore, it may be advantageous to provide a needle assembly having a distal tip section configured to prevent the removal of a free-floating tissue core from a biological wall as (or while) a pass-through hole is formed by the distal tip section.

To mitigate, at least in part, at least one problem associated with the existing technology, there is provided (in accordance with a major aspect) an apparatus. The apparatus includes and is not limited to (comprises) a needle assembly configured to be movable into a cavity of a patient having a biological wall. A distal tip section extends (distally) from the needle assembly. The distal tip section is configured to form a pass-through hole extending through the biological wall of the patient as (while) the needle assembly is urged to move toward the biological wall. The distal tip section is also configured to prevent the removal of a free-floating tissue core (which is depicted in the embodiment of FIG. 1) from the biological wall as (or while) the pass-through hole is formed by the distal tip section.

To mitigate, at least in part, at least one problem associated with the existing technology, there is provided (in accordance with a major aspect) an apparatus. The apparatus includes and is not limited to (comprises) a needle assembly configured to be movable into a cavity of a patient having a biological wall. A distal tip section extends (distally) from the needle assembly. The distal tip section surrounds a lumen entrance leading to a lumen extending interiorly along a length of the needle assembly. The distal tip section is configured to form a pass-through hole extending through the biological wall of the patient as (while) the needle assembly is urged to move toward the biological wall. The distal tip section is also configured to prevent (at least in part) the removal of a free-floating tissue core (which is depicted in the embodiment of FIG. 1, and which may result by assistance from, or the presence of, the lumen entrance from the biological wall as (or while) the pass-through hole is formed by the distal tip section.

To mitigate, at least in part, at least one problem associated with the existing technology, there is provided (in accordance with a major aspect) a method. The method is for forming a pass-through hole through a biological wall of a patient having a cavity. The method includes and is not limited to (comprises) moving a needle assembly into the cavity of the patient having the biological wall, in which the needle assembly includes a distal tip section extending (distally) from the needle assembly. The method also includes using the distal tip section to form the pass-through hole through the biological wall of the patient as (while) the needle assembly is urged to move toward the biological wall. The method includes using the distal tip section to prevent the removal of a free-floating tissue core (which is depicted in the embodiment of FIG. 1) from the biological wall as (or while) the pass-through hole is formed by the distal tip section.

To mitigate, at least in part, at least one problem associated with the existing technology, there is provided (in accordance with a major aspect) a method. The method is for forming a pass-through hole through a biological wall of a patient having a cavity. The method includes and is not limited to (comprises) moving a needle assembly into the cavity of the patient having the biological wall, in which the needle assembly includes a distal tip section extending (distally) from the needle assembly, and the distal tip section surrounding a lumen entrance leading to a lumen extending interiorly along a length of the needle assembly. The method also includes using the distal tip section to form the pass-through hole through the biological wall of the patient as (while) the needle assembly is urged to move toward the biological wall. The method also includes using the distal tip section to prevent the removal of a free-floating tissue core (which is depicted in the embodiment of FIG. 1) from the biological wall (that may result by assistance from, or the presence of, the lumen entrance) as (while) the pass-through hole is formed by the distal tip section.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 2, FIG. 3 and FIG. 4 depict side views of embodiments of a needle assembly having a distal tip section; and

FIG. 5 and FIG. 6 depict a perspective view (FIG. 5) and an end view (FIG. 6) of embodiments of the needle assembly of FIG. 2; and

FIG. 7, FIG. 8, FIG. 9 and FIG. 10 depict a cross-sectional view (FIG. 7) and side views (FIG. 8, FIG. 9 and FIG. 10) of embodiments of the needle assembly of FIG. 2; and

FIG. 11 and FIG. 12 depict perspective views of embodiments of the needle assembly of FIG. 2; and

FIG. 13 depicts a perspective view of an embodiment of the needle assembly of FIG. 2.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

| | |
|---|---|
| needle assembly 102 | distal tip section 104 |
| circumferential peripheral leading edge 105 | known needle assembly 802 |
| lumen entrance 106 | known distal tip section 804 |
| lumen 108 | known lumen 806 |
| electrically-conductive surface 110 | cavity 900 |
| dielectric surface 112 | patient 902 |
| safety cover 114 | biological wall 904 |
| extended section 116 | pass-through hole 905 |
| first arcuate portion 200 | free-floating tissue core 906 |
| second arcuate portion 202 | tissue flap 908 |

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

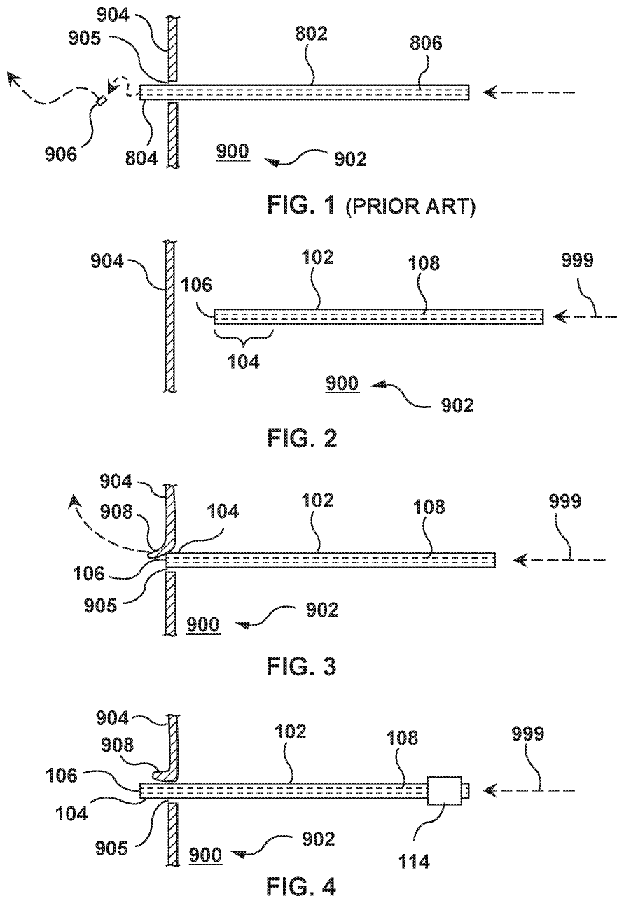

FIG. 2, FIG. 3 and FIG. 4 depict side views of embodiments of a needle assembly 102 having a distal tip section 104.

Referring to the embodiment as depicted in FIG. 2, the needle assembly 102 may be configured to be inserted into a confined space defined by the patient 902. The needle assembly 102 may be configured to guide the insertion of a medical instrument (known and not depicted, such as a catheter, etc. and any equivalent thereof) into the confined space defined by the patient 902. The needle assembly 102 includes, and is not limited to, an elongated flexible tube (made from a medical grade material) configured to be inserted into the patient 902. The needle assembly 102 is (preferably) impermeable by a bodily fluid of the patient 902. The needle assembly 102 includes (in accordance with another option) superelastic nitinol. Nitinol alloys exhibit two closely related and unique properties: shape memory effect (SME) and superelasticity (SE; also called pseudoelasticity or PE). Shape memory is the ability of nitinol to undergo deformation at one temperature, then recover its original, undeformed shape upon heating above its transformation temperature. Superelasticity occurs at a narrow temperature range just above its transformation temperature; in this case, no heating is necessary to cause the undeformed shape to recover, and the material exhibits enormous elasticity, from about ten (10) to thirty (30) times that of ordinary metal. The needle assembly 102 may include a shape memory material configured to be manipulated and/or deformed followed by a return to the original shape the material was set in. Shape memory materials (SMMs) are configured to recover their original shape from a significant and seemingly plastic deformation in response to a particular stimulus is applied to the material. This may be known as the shape memory effect (SME). Superelasticity (in alloys) may be observed if (when) the shape memory material is deformed at the present of the stimulus. The needle assembly 102 may include any bio-compatible material having properties suitable for sufficient performance properties (dielectric strength, thermal performance, insulation and corrosion, water and heat resistance) for safe performance to comply with industrial and regulatory safety standards (or compatible for medical usage). Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [published 2014].

Referring to the embodiments as depicted in FIG. 2, FIG. 3 and FIG. 4, there is depicted a major aspect of an apparatus. The apparatus includes and is not limited to (comprises) a needle assembly 102. The needle assembly 102 is configured to be movable into a cavity 900 of a patient 902 having a biological wall 904 (an internal biological wall). A distal tip section 104 extends (distally) from the needle assembly 102. The distal tip section 104 surrounds (at least in part) a lumen entrance 106. The lumen entrance 106 leads to a lumen 108. The lumen 108 extends (at least in part) interiorly along a length of the needle assembly 102. The distal tip section 104 is configured to form a pass-through hole 905 (in which the pass-through hole 905 extends, once formed, through the biological wall 904 of the patient 902) as (while) the needle assembly 102 is urged to move toward the biological wall 904. The distal tip section 104 is also configured to prevent (at least in part) the removal of a free-floating tissue core 906 (which is depicted in the embodiment of FIG. 1), which may result by assistance from, or the presence of, the lumen entrance 106, from the biological wall 904 (as, or while, the pass-through hole 905 is formed by the distal tip section 104).

Referring to the embodiments as depicted in FIG. 2, FIG. 3 and FIG. 4, the distal tip section 104 is (preferably) also configured to form a tissue flap 908 that remains attached to, and extends from, the biological wall 904 (preferably, as, or while, the pass-through hole 905 is formed by the distal tip section 104, in which the tissue flap 908 (as depicted in FIG. 4) is positioned proximate to the pass-through hole 905).

Referring to the embodiments as depicted in FIG. 2, FIG. 3 and FIG. 4, there is depicted a major aspect of a method. The method is for forming a pass-through hole 905 through a biological wall 904 of a patient 902 having a cavity 900. The method includes and is not limited to (comprises) a first operation including moving a needle assembly 102 into the cavity 900 of the patient 902; the needle assembly 102 includes a distal tip section 104 extending (distally) from the needle assembly 102; the distal tip section 104 surrounds a lumen entrance 106 leading to a lumen 108 extending interiorly along a length of the needle assembly 102. The method also includes and is not limited to (comprises) a second operation including using the distal tip section 104 to form the pass-through hole 905 through the biological wall 904 of the patient 902 as (while) the needle assembly 102 is urged to move toward the biological wall 904. The method also includes and is not limited to (comprises) a third operation including using the distal tip section 104 to prevent the removal of a free-floating tissue core 906 (which is depicted in the embodiment of FIG. 1) from the biological wall 904 (which may result by assistance from, or the presence of, the lumen entrance 106 (as, or while, the pass-through hole 905 is formed by the distal tip section 104).

Referring to the embodiments as depicted in FIG. 2, FIG. 3 and FIG. 4, the needle assembly 102, for instance, may allow a user (surgeon) to perform a transseptal puncture across the fossa ovalis in the heart of the patient 902.

Referring to the embodiments as depicted in FIG. 2, FIG. 3 and FIG. 4, the needle assembly 102 includes (preferably) SAE (Society of Automotive Engineers) number 304 stainless steel containing chromium (between about 15% and about 20%) and nickel (between about 2% and about 10.5%). The needle assembly 102 is (preferably) made of an electrically conductive material, and offers a stiffness profile that is suitable for surgical procedures. It will be appreciated that any conductive material may be used for the needle assembly 102.

Referring to the embodiments as depicted in FIG. 2, FIG. 3 and FIG. 4, a molded plastic handle (known and not depicted) may be positioned at a proximal end of the needle assembly 102. The molded plastic handle may allow for manipulation of the needle assembly 102 at the proximal end of the needle assembly 102. It will be appreciated that the handle adds convenience for the user.

Referring to the embodiments as depicted in FIG. 2, FIG. 3 and FIG. 4, a cable-facilitating connection (known and not depicted) is configured to electrically connect the distal tip section 104 (via the needle assembly 102) to a radiofrequency energy generator (known and not depicted). A cable is configured to facilitate an electrical connection to the needle assembly 102 for conveying radiofrequency energy (from the radiofrequency energy generator) to the needle assembly 102 to the biological wall 904 (target tissue) at the distal tip section 104.

Referring to the embodiments as depicted in FIG. 2, FIG. 3 and FIG. 4, the overall length of the needle assembly 102 may be compatible with conventional transseptal sheaths and dilators (known and not depicted). This facilitates the improved usability of the needle assembly 102 across a variety of accessory devices that the user may be free to choose from. For instance, the overall length of the needle assembly 102 may be about 71 centimeters (cm), about 89 cm or about 98 cm, etc.

Referring to the embodiments as depicted in FIG. 2, FIG. 3 and FIG. 4, the needle assembly 102 has (preferably) a distal section diameter that is compatible with conventional transseptal accessory devices (known and not depicted). The distal section of the needle assembly 102 (preferably) does not exceed about 0.032 inches in diameter. Alternatively, the distal section of the needle assembly 102 does not exceed about 0.035 inches in diameter.

Referring to the embodiments as depicted in FIG. 2, FIG. 3 and FIG. 4, the needle assembly 102 may have an overall length that is compatible with conventional transseptal accessory devices. The overall length of the needle assembly 102 may be any length. The needle assembly 102 may be of a length where the needle assembly 102 is able to reach the fossa ovalis of the inter atrial septum of the heart of the patient 902 (from wherever a user has accessed the vasculature percutaneously).

Referring to the embodiments as depicted in FIG. 2, FIG. 3 and FIG. 4, the needle assembly 102 may have a distal section diameter that may be any suitable size, and/or may not exceed a diameter where blood flow in the vasculature, which the needle assembly 102 travels through, is excessively impeded.

Referring to the embodiments as depicted in FIG. 2, FIG. 3 and FIG. 4, while a circular profile of the needle assembly 102 and/or the distal tip section 104 may provide the greatest compatibility with existing accessory devices, the needle assembly 102 may have any suitable shape.

Referring to the embodiments as depicted in FIG. 2, FIG. 3 and FIG. 4, another option may include the following: instead of the needle assembly 102 being made of an electrically-conductive material with the dielectric surface 112 (a dielectric coating) covering at least one or more sections of the distal tip section 104, the materials may be reversed. The needle assembly 102 may be constructed where the needle assembly 102 is made up of an electrically insulating material and a portion of the distal tip section 104 is made of an electrically conductive material. The distal tip section 104 having a conductive material may be connected to a known device configured to generate radiofrequency energy via a conduit (wire) of conductive material that is internal (or external or both) to the needle assembly 102.

Referring to the embodiments as depicted in FIG. 2, FIG. 3 and FIG. 4, the needle assembly 102 may include a radiofrequency needle constructed with a flap on a distal end made of a non-conductive material that closes the open lumen when (once) radiofrequency energy is applied thereto. This arrangement may prevent tissue coring (as depicted in FIG. 1) since there is no vaporization of tissue along a closed circumferential pathway that may cause division of tissue into multiple segments. Following application of radiofrequency energy, the flap may be moved to a position where it is no longer occluding the lumen 108 of the needle assembly 102.

Referring to the embodiments as depicted in FIG. 2, FIG. 3 and FIG. 4, the needle assembly 102 may include a radiofrequency needle constructed with a closed distal end used to apply radiofrequency energy to a target tissue. A lumen is exposed on the side of the needle assembly 102 near the distal tip section 104. The lumen 108 may facilitate functionality such as fluid delivery and aspiration, wire anchoring, and pressure measurements. Coring of tissue (as depicted in FIG. 1) may be avoided since the distal tip section 104 (once activated where radiofrequency energy is applied thereto) is not a circumferential profile that may cause division of tissue into multiple segments.

Referring to the embodiments as depicted in FIG. 2, FIG. 3 and FIG. 4, a radiofrequency needle is (preferably) constructed where the open lumen at the distal tip consists of an electrically conductive material that is discontinuous. It is discontinuous in that it does not create a fully enclosed perimeter of electrically conductive material at the distal end. Such a construct would require a mechanism to ensure that when the needle passes through tissue beyond the discontinuous distal end, the needle is no longer electrically active and thus cannot core tissue.

Referring to the embodiment as depicted in FIG. 4, rather than the needle assembly 102 (also called a puncture device) with the lumen entrance 106 leading to the lumen 108, an open-lumen cannula system with an accessory radiofrequency guidewire (known and not depicted) may be provided. The open-lumen cannula system may provide a conduit to a desired puncture location (such as the pass-through hole 905) on the fossa ovalis of the inter atrial septum in the heart of the patient 902. Through the conduit, a guidewire (known and not depicted) may be advanced to the desired puncture location. The guidewire may have a core of conductive material surrounded by electrical insulation except at the distal tip and the proximal end in order to facilitate connection to a device (known and not depicted) that generates radiofrequency energy. Through the guidewire, radiofrequency energy may be applied to the pass-through hole 905, vaporizing tissue without tissue coring (as depicted in FIG. 1).

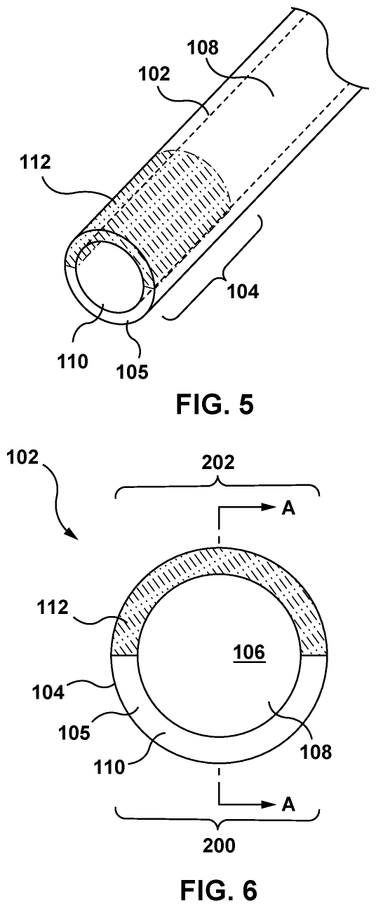

FIG. 5 and FIG. 6 depict a perspective view (FIG. 5) and an end view (FIG. 6) of embodiments of the needle assembly 102 of FIG. 2.

Referring to the embodiment as depicted in FIG. 5 (a perspective view), the needle assembly 102 defines (has) the lumen 108 (extending along an elongated length of the needle assembly 102). The needle assembly 102 includes (is made of), preferably, an electrically conductive material. The needle assembly 102, preferably, includes an electrically-conductive surface 110. The needle assembly 102, preferably, also includes a dielectric surface 112 (also called a dielectric coating) that covers a portion or a part of the distal tip section 104 (such as, a portion of the circumference at the distal tip section 104). The dielectric surface 112 provides electrical insulation to at least one or more selected regions of the distal tip section 104 (so that touching tissue with the dielectric surface 112 does not become vaporized); therefore, the tissue that is touched by the dielectric surface 112 does not become fully separated during a puncture procedure associated with utilization of the needle assembly 102. Rather than forming the free-floating tissue core 906 (which is depicted in the embodiment of FIG. 1) by using the known needle assembly 802 (as depicted in FIG. 1), the dielectric surface 112 may avoid the formation of the free-floating tissue core 906 altogether. For instance, the dielectric surface 112 may, as an option or alternative to the embodiment depicted in FIG. 1, assist in the formation of the tissue flap 908 (as depicted in FIG. 4) while the pass-through hole 905 is formed by movement of the distal tip section 104 through the biological wall 904 (so that a portion of the needle assembly 102 may pass through the biological wall 904. The embodiment of FIG. 5 avoids the formation of the free-floating tissue core 906, as depicted in FIG. 1 (that is, tissue coring does not occur).

Referring to the embodiments as depicted in FIG. 4 and FIG. 5, a safety cover 114 (as depicted in FIG. 4) is (preferably) applied to cover an outer surface of the needle assembly 102. The safety cover 114 includes an electrically-insulated material. The safety cover 114 covers a remainder of the needle assembly 102 except for the distal tip section 104. The safety cover 114 may include a heat shrink material, etc. The safety cover 114 includes (preferably) a heat shrink having polytetrafluoroethylene (PTFE), which may offer a relatively greater lubricity compared to parylene, which may ease delivery into and removal from an accessory device (known and not depicted) and/or patient vasculature (the vascular system of a part of the body and its arrangement). It is preferred that only a relatively small section at the distal tip section 104 is coated with the dielectric surface 112 (as depicted in FIG. 5) while the remainder of the needle assembly 102 is covered with the safety cover 114 (heat shrink). It will be appreciated that the safety cover 114 may include any electrically insulating material, and/or may include the dielectric coatings described in this document.

Referring to the embodiments as depicted in FIG. 4 and FIG. 5, the dielectric surface 112 (a dielectric coating) partially covers a circumference of the distal tip section 104 and/or the circumferential peripheral leading edge 105 of the distal tip section 104. The uncoated portion (electrically-active portion or the electrically-conductive surface 110) of the distal tip section 104 is used to vaporize tissue (as depicted in FIG. 4) at a desired portion of the biological wall 904 (such as, the fossa ovalis of the heart) once the electrically-conductive surface 110 is activated or energized (such as when radiofrequency energy is transmitted to a portion of the biological wall 904 via the electrically-conductive surface 110). Tissue coring (as depicted in FIG. 1) is mitigated by the dielectric surface 112 (the dielectric coating) which prevents vaporization of tissue (a portion of the biological wall 904, as depicted in FIG. 4). Preventing cutting by (such as, vaporization) at least one section (or some sections) of the distal tip section 104 (or of the circumference of the distal tip section 104), separation of the tissue (that is, tissue coring, as depicted in FIG. 1) may be avoided or prevented at least in part (for the case where the electrically-conductive surface 110 is activated, such as when radiofrequency energy is applied to the electrically-conductive surface 110, etc.).

Referring to the embodiment as depicted in FIG. 5, the distal tip section 104 (preferably) includes a radiofrequency tip assembly (known by those skilled in the art, and not depicted). An embodiment of the radiofrequency may include the NRG (TRADEMARK) RF Transseptal Needle manufactured by Baylis Medical (headquartered in Canada), and is configured to assist the physician in gaining access to the left atrium by using radiofrequency (RF) energy in a controlled manner as opposed to mechanical force.

Referring to the embodiment as depicted in FIG. 5, the needle assembly 102 is made of an electrically conductive material. A dielectric coating covers a portion of the electrically conductive material. The distal tip section 104 (preferably) is configured to puncture the biological wall 904 (also called tissue) via utilizing radiofrequency energy. The lumen 108 extends through the distal tip section 104 that defines the lumen entrance 106 extending from the lumen 108. The distal tip section 104 (preferably) has a profile that is partially coated by the dielectric surface 112. This partial coating (of the dielectric surface 112) is configured to mitigate tissue coring (which is depicted in the embodiment of FIG. 1). A part of the distal tip section 104 may have a portion (of an electrically active perimeter) of electrically conductive material (the electrically-conductive surface 110) positioned at the distal tip section 104. Part of the perimeter of the distal tip section 104 is dielectrically coated (with the dielectric surface 112) to avoid separation of tissue (as depicted in FIG. 1) once radiofrequency energy is applied to the distal tip section 104.

Referring to the embodiment as depicted in FIG. 5, the needle assembly 102 may include (define) a lumen entrance 106 leading into the lumen 108. The lumen entrance 106 is positioned at the distal tip section 104. A dielectric coating (the dielectric surface 112) is applied to at least one or more sections of the distal tip section 104 (that surround the lumen entrance 106 at the distal tip section 104) to prevent tissue coring (as depicted in the embodiment of FIG. 1). The dielectric coating (the dielectric surface 112) may cover at least part of the perimeter surrounding the lumen entrance 106 defined at the distal tip section 104.

Referring to the embodiments as depicted in FIG. 5, an exposed electrically-conductive surface 110 and an exposed dielectric surface 112 are configured to cover different portions (sections) of the distal tip section 104.

Referring to the embodiments as depicted in FIG. 5, the exposed dielectric surface 112 is positionable proximate to (adjacent to) the exposed electrically-conductive surface 110.

Referring to the embodiments as depicted in FIG. 5, the exposed electrically-conductive surface 110 and the exposed dielectric surface 112 are each configured to make contact with, at least in part, the biological wall 904 (preferably as or while the needle assembly 102 is urged to move toward the biological wall 904, as depicted in the embodiment of FIG. 4).

Referring to the embodiment as depicted in FIG. 5, the dielectric surface 112 includes (preferably) a chemical vapor deposited poly(p-xylylene) polymer configured to provide a moisture and dielectric barrier, such as parylene (TRADEMARK). The dielectric surface 112 (dielectric coating) partially covers the distal tip section 104. The thickness of the dielectric coating may be such that dielectric breakdown does not occur under the electrical parameters utilized by, for instance, a radiofrequency generator that the distal tip section 104 is configured to interact therewith. The thickness of the dielectric coating, for instance, may be at least about 30 microns, and a final appropriate coating thickness may be dependent on the electrical parameters used for the needle assembly 102. It will be appreciated that any material of sufficient dielectric strength to prevent the transmission of electricity (such as radiofrequency energy) to a portion of the circumference of the distal tip section 104 may be acceptable. It will be appreciated that any material of sufficient dielectric strength to prevent the transmission of electricity (radiofrequency energy) to the covered portions of the distal tip section 104 may be acceptable. Further, the dielectric coating may be applied, for instance, to an interior of the lumen entrance 106 and/or the lumen 108.

Referring to the embodiment as depicted in FIG. 5, the dielectric surface 112 has (preferably) a thickness of about 30 micron of parylene. The dielectric surface 112 may include other suitable coatings or materials, such as silicon dioxide, aluminum oxide, any alternative formulation of parylene, a silicone-based coating formulation, a fluoropolymer coating, etc., and any equivalent thereof.

Referring to the embodiment as depicted in FIG. 5, in accordance with one option, the dielectric surface 112 is applied to an exterior portion of the distal tip section 104, which may be sufficient to prevent tissue coring (as depicted in FIG. 1).

Referring to the embodiment as depicted in FIG. 5, in accordance with another option, the dielectric surface 112 may be applied to an inside surface of the lumen entrance 106 and/or the lumen 108. While the needle assembly 102 is made to pass through the tissue (that is, the biological wall 904, as depicted in FIG. 4), the interior of the needle assembly 102 comes into proximity with the biological wall 904 (tissue). While the inside surface of the lumen entrance 106 and/or the lumen 108 may not necessarily directly touch the biological wall 904, the proximity of the inner surface may be close enough that electrical arcing may occur and inadvertently cut the tissue (this condition may not be desirable) and thus may result in tissue coring (as depicted in FIG. 1). To mitigate such a condition, a length, such as about five (5) millimeters (mm), of the interior surface of the lumen entrance 106 and/or the lumen 108 may be coated with the dielectric surface 112 that may prevent unwanted (electrical) arcing that may occur from the interior of the distal tip section 104 as the distal tip section 104 of the needle assembly 102 is passed through the biological wall 904 (as depicted in FIG. 4).

Referring to the embodiment as depicted in FIG. 6 (an end view), a circumferential profile (of the distal tip section 104) is depicted. The distal tip section 104 includes (preferably) a first arcuate portion 200 and a second arcuate portion 202 positioned proximate to the first arcuate portion 200. The distal tip section 104 includes (preferably) a circumferential peripheral leading edge 105. The circumferential peripheral leading edge 105 includes (preferably) a first arcuate portion 200 and a second arcuate portion 202.

Referring to the embodiment as depicted in FIG. 6, a portion (the dielectric surface 112) of the distal tip section 104 is coated in (with) a dielectric material configured to provide electrical insulation. For instance, once radiofrequency energy is applied to the distal tip section 104, the uncoated portion (the active portion which is not coated with the dielectric material) of the distal tip section 104 is activated (energized) and may cut tissue (the biological wall 904) while the portion that is coated with the dielectric material cannot (does not) cut tissue. As a result, there is no separation of tissue that occurs (as is the case with the embodiment of FIG. 1).

Referring to the embodiments as depicted in FIG. 6, the distal tip section 104 includes a first arcuate portion 200 and a second arcuate portion 202 being positioned proximate to the first arcuate portion 200. The exposed electrically-conductive surface 110 is also configured to cover the first arcuate portion 200. The exposed dielectric surface 112 is also configured to cover (covers) the second arcuate portion 202 of the distal tip section 104.

Referring to the embodiments as depicted in FIG. 6, the exposed electrically-conductive surface 110 is also configured to electrically cut through the biological wall 904 (preferably, once the exposed electrically-conductive surface 110 of the distal tip section 104, in use, is moved to make contact, at least in part, with the biological wall 904, and as or while the exposed electrically-conductive surface 110 is activated for cutting through the biological wall 904).

Referring to the embodiments as depicted in FIG. 6, the exposed dielectric surface 112 is also configured to electrically isolate a portion of the distal tip section 104 surrounding the lumen entrance 106 from the biological wall 904; this is done, preferably, as (or while) the exposed electrically-conductive surface 110, in use, makes contact with the biological wall 904, and as the exposed electrically-conductive surface 110 is activated to form a pass-through hole 905 extending through the biological wall 904 (and while the needle assembly 102 is urged to move toward the biological wall 904, as depicted in the embodiment of FIG. 4).

Referring to the embodiments as depicted in FIG. 6, the exposed dielectric surface 112 is configured to electrically isolate a portion of the distal tip section 104 surrounding the lumen entrance 106 from the biological wall 904 (while the exposed electrically-conductive surface 110 is activated, and the exposed dielectric surface 112 avoids formation of the free-floating tissue core 906 that is depicted in the embodiment of FIG. 1).

Referring to the embodiments as depicted in FIG. 6, the distal tip section 104 presents (has) a circumferential peripheral leading edge 105 surrounding the lumen entrance 106. The circumferential peripheral leading edge 105 is configured to prevent the removal of a free-floating tissue core 906 from the biological wall 904 (as, or while, the pass-through hole 905 is formed by the distal tip section 104).

Referring to the embodiments as depicted in FIG. 6, an exposed electrically-conductive surface 110 is configured to cover (covers) a first arcuate portion 200 of the circumferential peripheral leading edge 105. An exposed dielectric surface 112 is configured to cover (covers) a second arcuate portion 202 of the circumferential peripheral leading edge 105. The second arcuate portion 202 is positioned proximate to the first arcuate portion 200. The exposed dielectric surface 112 is positionable proximate to the exposed electrically-conductive surface 110. The exposed electrically-conductive surface 110 and the exposed dielectric surface 112 are each configured to make contact with, at least in part, the biological wall 904 (as, or while, the needle assembly 102, in use, is moved toward the biological wall 904).

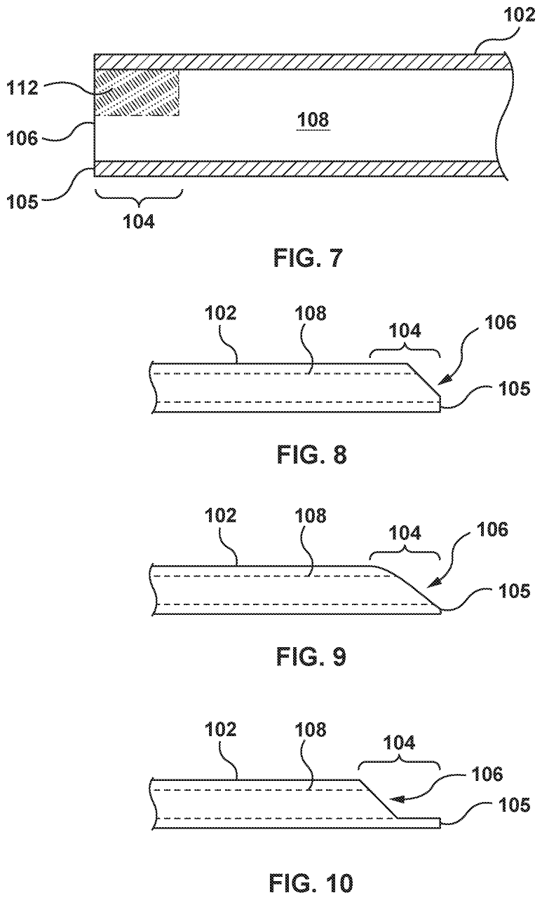

FIG. 7, FIG. 8, FIG. 9 and FIG. 10 depict a cross-sectional view (FIG. 7) and side views (FIG. 8, FIG. 9 and FIG. 10) of embodiments of the needle assembly 102 of FIG. 2. The view depicted in FIG. 7 is taken along a cross-sectional line A-A of FIG. 6.

Referring to the embodiment as depicted in FIG. 7 (a cross-sectional view), the dielectric surface 112 (also called a dielectric coating or electrical insulation) is applied to an interior surface facing the lumen 108 and/or the lumen entrance 106. This arrangement mitigates the unwanted (electrical) arcing that may occur when tissue (the biological wall 904, as depicted in FIG. 4) passes near the distal tip section 104 (which may result in tissue coring as depicted in FIG. 1).

Referring to the embodiments as depicted in FIG. 8, FIG. 9 and FIG. 10, there are depicted various embodiments of side profiles of the distal tip section 104. In some embodiments, the distal tip section 104 has a tapered profile configured to ease crossing through the biological wall 904 (as depicted in FIG. 4). Initially, a cross-sectional section that is smaller than the overall diameter of the distal tip section 104 passes through the biological wall 904 (the wall of tissue), then the crossing site is incrementally dilated wider to a relatively fuller diameter of the distal tip section 104 as the tapered section moves through the biological wall 904 (the crossing site).

Referring to the embodiment as depicted in FIG. 8, the distal tip section 104 presents a sloped leading surface.

Referring to the embodiment as depicted in FIG. 9, the distal tip section 104 provides a blunted portion with a tapered profile. The blunted distal tip mitigates for scraping of a plastic material (skiving) that may occur as the needle assembly 102 is advanced through an accessory device (known to persons of skill in the art and not depicted). The tapered profile is configured to enhance crossing through the biological wall 904 (as depicted in FIG. 4), such as the septum crossing efficacy, as the size of hole (the pass-through hole 905, as depicted in FIG. 4) initially created via puncture (by utilization of the needle assembly 102) is smaller compared to the overall diameter of the distal tip section 104. The tapered profile is configured to allow the puncture hole (the pass-through hole 905) to be expanded gradually up to a relatively larger diameter, generating the sensation of smoothness rather than an abrupt jump when crossing the biological wall 904 (such as the inter atrial septum of the heart, etc.). It will be appreciated that any profile (outer shape) of the distal tip section 104 may be utilized. The distal tip section 104, preferably, provides or defines the lumen entrance 106 leading to the lumen 108 positioned within the needle assembly 102.

Referring to the embodiment as depicted in FIG. 10, the distal tip section 104 presents an extension portion that extends ahead of the sloped leading surface.

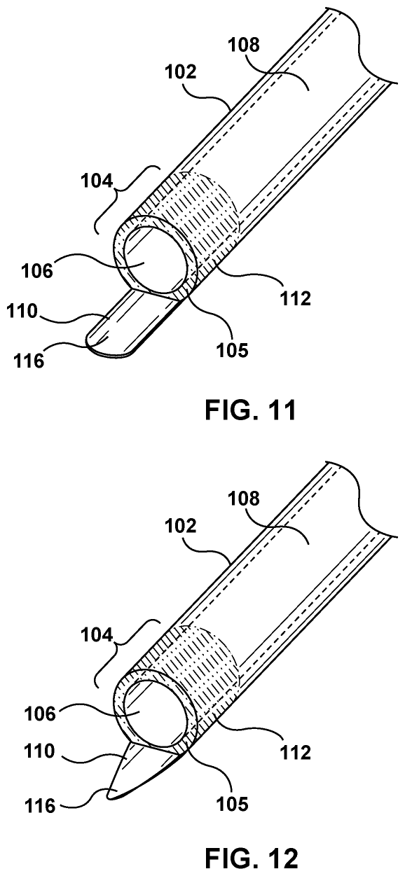

FIG. 11 and FIG. 12 depict perspective views of embodiments of the needle assembly 102 of FIG. 2.

Referring to the embodiments as depicted in FIG. 11 and FIG. 12, an extended section 116, of the distal tip section 104, extends (forwardly) from the distal tip section 104. The extended section 116 is made of (includes) an electrically-conductive surface 110, and the remainder of the profile of the distal tip section 104 (or the circumferential peripheral leading edge 105) is coated in a dielectric material (that is, the dielectric surface 112 includes the remainder of the profile of the distal tip section 104 or the circumferential peripheral leading edge 105). For instance, there may be an advantage to prevent radiofrequency energy from being applied to tissue (at the biological wall 904) at these regions.

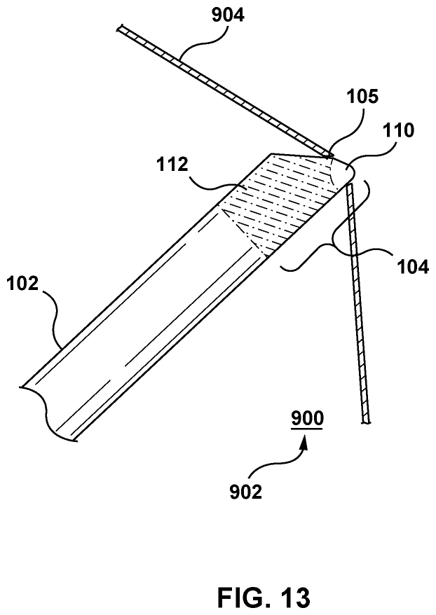

FIG. 13 depicts a perspective view of an embodiment of the needle assembly 102 of FIG. 2.

Referring to the embodiment as depicted in FIG. 13, the distal tip section 104 is depicted crossing the biological wall 904. Radiofrequency energy is applied (via the electrically-conductive surface 110) to the tissue at the uncoated portion of the distal tip section 104 while the remainder of the sections of the needle assembly 102 in close proximity to tissue are covered in the dielectric coating (forming the dielectric surface 112 thereon). Tissue is only vaporized at the electrically-conductive surface 110 (that is, the uncoated portion of the distal tip section 104). In accordance with the embodiment of FIG. 13, the needle assembly 102 does not provide or include the lumen entrance 106 and/or the lumen 108 (which are items depicted in the embodiment of FIG. 2, for instance).

Referring to the embodiment as depicted in FIG. 13, there is depicted a major aspect of an apparatus. The apparatus includes and is not limited to (comprises) a needle assembly 102. The needle assembly 102 is configured to be movable into a cavity 900 of a patient 902 having a biological wall 904 (an internal biological wall). A distal tip section 104 extends (distally) from the needle assembly 102. The distal tip section 104 is configured to form a pass-through hole 905 extending through the biological wall 904 of the patient 902 as (while) the needle assembly 102 is urged to move toward the biological wall 904 (as depicted in the embodiment of FIG. 4). The distal tip section 104 is also configured to prevent the removal of a free-floating tissue core 906 (that is depicted in the embodiment of FIG. 1) from the biological wall 904 as (or while) the pass-through hole 905 is formed by the distal tip section 104.

Referring to the embodiment as depicted in FIG. 13, an exposed electrically-conductive surface 110 and an exposed dielectric surface 112 are configured to cover different portions of the distal tip section 104.

Referring to the embodiment as depicted in FIG. 13, the exposed dielectric surface 112 is positionable proximate to (adjacent to) the exposed electrically-conductive surface 110.

Referring to the embodiment as depicted in FIG. 13, the exposed electrically-conductive surface 110 and the exposed dielectric surface 112 are each configured to make contact with, at least in part, the biological wall 904 as (or while) the needle assembly 102 is urged to move toward the biological wall 904 (as depicted in the embodiment of FIG. 4).

Referring to the embodiments as depicted in FIG. 13, there is depicted a major aspect of a method. The method is for forming a pass-through hole 905 through a biological wall 904 of a patient 902 having a cavity 900. The method includes and is not limited to (comprises) a first operation, including moving a needle assembly 102 into the cavity 900 of the patient 902 having the biological wall 904. The needle assembly 102 includes a distal tip section 104 extending (distally) from the needle assembly 102. The method includes and is not limited to (comprises) a first operation, including using the distal tip section 104 to form the pass-through hole 905 through the biological wall 904 of the patient 902 as (while) the needle assembly 102 is urged to move toward the biological wall 904 (as depicted in the embodiment of FIG. 4). The method includes and is not limited to (comprises) a first operation, including using the distal tip section 104 to prevent the removal of a free-floating tissue core 906 (that is depicted in the embodiment of FIG. 1) from the biological wall 904 as (or while) the pass-through hole 905 is formed by the distal tip section 104.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
a needle assembly being configured to be movable into a cavity of a patient having a biological wall; and
a distal tip section extending from the needle assembly; and
the distal tip section being configured to form a pass-through hole extending through the biological wall of the patient as the needle assembly is urged to move toward the biological wall; and
the distal tip section also being configured to prevent, at least in part, removal of a free-floating tissue core from the biological wall as the pass-through hole is formed by the distal tip section.

2. The apparatus of claim 1, further comprising:
an exposed electrically conductive surface and an exposed dielectric surface being configured to cover different portions of the distal tip section.

3. The apparatus of claim 2, wherein:
the exposed dielectric surface is positionable proximate to the exposed electrically conductive surface.

4. The apparatus of claim 2, wherein:
the exposed electrically conductive surface and the exposed dielectric surface are each configured to make contact with, at least in part, the biological wall as the needle assembly is urged to move toward the biological wall.

5. An apparatus, comprising:
a needle assembly being configured to be movable into a cavity of a patient having a biological wall; and
a distal tip section extending from the needle assembly, and the distal tip section surrounding a lumen entrance leading to a lumen extending interiorly along a length of the needle assembly; and
the distal tip section being configured to form a pass-through hole extending through the biological wall of the patient as the needle assembly is urged to move toward the biological wall; and
the distal tip section also being configured to prevent, at least in part, removal of a free-floating tissue core, which may result by assistance from, or presence of, the lumen entrance, from the biological wall as the pass-through hole is formed by the distal tip section.

6. The apparatus of claim 5, wherein:
the distal tip section is also configured to form a tissue flap that remains attached to, and extends from, the biological wall as the pass-through hole is formed by the distal tip section, in which the tissue flap is positioned proximal to the pass-through hole.

7. The apparatus of claim 5, further comprising:
an exposed electrically conductive surface and an exposed dielectric surface being configured to cover different portions of the distal tip section.

8. The apparatus of claim 7, wherein:
the exposed dielectric surface is positionable proximate to the exposed electrically conductive surface.

9. The apparatus of claim 7, wherein:
the exposed electrically conductive surface and the exposed dielectric surface are each configured to make contact with, at least in part, the biological wall as the needle assembly is urged to move toward the biological wall.

10. The apparatus of claim 7, wherein:
the distal tip section includes a first arcuate portion and a second arcuate portion being positioned proximal to the first arcuate portion; and
the exposed electrically conductive surface is also configured to cover the first arcuate portion; and
the exposed dielectric surface is also configured to cover the second arcuate portion of the distal tip section.

11. The apparatus of claim 7, wherein:
the exposed electrically-conductive surface is also configured to electrically cut through the biological wall once the exposed electrically-conductive surface of the distal tip section, in use, is moved to make contact, at least in part, with the biological wall and is activated for cutting through the biological wall.

12. The apparatus of claim 7, wherein:
the exposed dielectric surface is also configured to electrically isolate a portion of the distal tip section surrounding the lumen entrance from the biological wall as the exposed electrically-conductive surface, in use, makes contact with the biological wall, and as the exposed electrically-conductive surface is activated to form a pass-through hole extending through the biological wall while the needle assembly is urged to move toward the biological wall.

13. The apparatus of claim 7, wherein:
the exposed dielectric surface is configured to electrically isolate a portion of the distal tip section surrounding the lumen entrance from the biological wall while the exposed electrically-conductive surface is activated, and the exposed dielectric surface avoids formation of the free-floating tissue core.

14. The apparatus of claim 5, wherein:
the distal tip section presents a circumferential peripheral leading edge surrounding the lumen entrance; and
the circumferential peripheral leading edge is configured to prevent, at least in part, removal of a free-floating tissue core from the biological wall as the pass-through hole is formed by the distal tip section.

15. The apparatus of claim 14, further comprising:
an exposed electrically-conductive surface configured to cover a first arcuate portion of the circumferential peripheral leading edge.

16. The apparatus of claim 15, further comprising:
an exposed dielectric surface configured to cover a second arcuate portion of the circumferential peripheral leading edge, in which the second arcuate portion is positioned proximal to the first arcuate portion.

17. The apparatus of claim 16, wherein:
the exposed dielectric surface is positionable proximate to the exposed electrically-conductive surface.

18. The apparatus of claim 17, wherein:
the exposed electrically-conductive surface and the exposed dielectric surface each being configured to make contact with, at least in part, the biological wall as the needle assembly, in use, is moved toward the biological wall.

19. A method for forming a pass-through hole through a biological wall of a patient having a cavity, the method comprising:
moving a needle assembly into the cavity of the patient having the biological wall, in which the needle assembly includes a distal tip section extending from the needle assembly; and
using the distal tip section to form the pass-through hole through the biological wall of the patient as the needle assembly is urged to move toward the biological wall; and
using the distal tip section to prevent, at least in part, removal of a free-floating tissue core from the biological wall as the pass-through hole is formed by the distal tip section.

20. (canceled)
